(19) United States
(12) Patent Application Publication
(10) Pub. No.: US 2008/0262006 A1
(43) Pub. Date: Oct. 23, 2008

(54) Selective endothelin type-a antagonists

(76) Inventor: Scott L. Harbeson, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/012,508

(22) Filed: Feb. 1, 2008

Related U.S. Application Data
(60) Provisional application No. 60/887,942, filed on Feb. 2, 2007.
(60) Provisional application No. 60/976,564, filed on Oct. 1, 2007.

(51) Int. Cl. A61K 31/505; C07D 239/60; A61P 9/12

(52) U.S. Cl. 514/274; 544/302

(57) ABSTRACT

This invention relates to novel endothelin receptor antagonists that selectively inhibit the interaction between Endothelin-1 (ET-1) and endothelin type-A receptors, their derivatives, acceptable acid addition salts. This invention also provides compositions comprising a compound of this invention and the use of such compositions in methods of treating diseases and conditions that are beneficially treated by endothelin receptor antagonists, particularly those diseases and conditions that are beneficially treated by selective inhibitors of endothelin type-A receptors.

### RELATED APPLICATIONS

This application claims the benefit of U.S. Provisional Application No. 60/887,942, filed on Feb. 2, 2007 and U.S. Provisional Application No. 60/976,564, filed on Oct. 1, 2007. The entire teachings of the above applications are incorporated herein by reference.

### BACKGROUND OF THE INVENTION

Ambrisentan is known by the chemical name (S)-2-(4,6-dimethylpyrimidin-2-yloxy)-3-methoxy-3,3-diphenylpropanoic acid. Ambrisentan is known as Letairis®. Darusentan is known by the chemical name (S)-2-(4,6-dimethoxypyrimidin-2-yloxy)-3-methoxy-3,3-diphenylpropanoic acid.

Both ambrisentan and darusentan inhibit the interaction between Endothelin-1 (ET-1) and its receptors, which in turn suppresses the effects recruited by ET-1. ET-1 is a 21-amino acid peptide neurohormone, which was first isolated and described in 1988 and is an extremely potent and long-acting vasoconstrictor (Yanagisawa M et al, Nature 1988, 332: 418). ET-1 causes vasoconstriction by binding to receptors in the endothelium and vascular smooth muscle. ET-1 levels are elevated in the plasma and lung tissue of patients with pulmonary arterial hypertension, which suggests that ET-1 has a pathogenic role in this disease. There are two naturally occurring receptors for ET-1 designated ET_{A} and ET_{B}. When ET-1 binds to ET_{A} receptors, it causes vasoconstriction, whereas when ET-1 binds to ET_{B} receptors, it leads to the production of nitric oxide and prostacyclin, which in turn causes vasodilatation, the widening and relaxing of the blood vessels. Accordingly, the dual roles of ET_{A} and ET_{B} serve to counterbalance one another in healthy humans: the binding of ET-1 to ET_{A} causes vasoconstriction, while ET_{B} is thought to protect against excessive vasoconstriction.

Pulmonary arterial hypertension is a disease characterized by excessive constriction of the blood vessels in the lungs. Such constriction leads to high pulmonary arterial pressures, making it difficult for the heart to pump blood through the lungs. Since blood is oxygenated in the lungs, this constriction prevents the lungs from adequately oxygenating the blood, which causes persons afflicted with pulmonary arterial hypertension to suffer from extreme shortness of breath as the heart struggles to oxygenate the blood by pumping against high arterial pressures.

Mild pulmonary arterial hypertension is often treated with calcium channel blockers, diuretics and anticoagulants, which serve to lower blood pressure (Galie, N, Eur Cardiovasc Dis 2006, 32). Prior to 2001, more severe cases were treated with continuous intravenous infusion of prostacyclin. However, progress in the area of prostacyclins has lead to the development of more stable derivatives, which are available for subcutaneous infusion and inhalation.

The development of endothelin antagonists marked a significant advance in the pharmaceutical treatment of pulmonary arterial hypertension. Endothelin antagonists are effective in treating persons afflicted with moderate to severe pulmonary arterial hypertension. One such compound is N-[6-(2-hydroxyethoxy)-5-(2-methoxyphenoxy)-2-pyrimidin-2-yl-pyrimidin-4-yl]-4-tert-butyl-benzenesulfonamide, also known as Bosentan. The Food and Drug Administration (FDA) approved Bosentan for the treatment of PAH in 2001. This compound has been shown to be effective in decreasing the constriction of the pulmonary artery, thereby increasing the supply of blood to the lungs and reducing the workload incurred by the heart.

Ambrisentan and darusentan are believed to work by binding to endothelin receptor sites. Ambrisentan and darusentan selectively bind to ET_{A} receptor sites in the endothelium and vascular smooth muscle, hence showing a far greater affinity for ET_{A} than for ET_{B} (Riechers H et al, J Med Chem 1996, 39(11):2123).

Ambrisentan has been approved and launched in the US, is awaiting approval in the EU (the MAA is under review by the EMEA), and is in clinical trials in Japan, Canada, Australia, Israel and South America for the treatment of pulmonary arterial hypertension. Specific trials have investigated the use of ambrisentan in patients who have previously discontinued use of an alternative endothelin antagonist, such as Bosentan or Sitaxsentan, due to liver function test abnormalities. Ambrisentan has been given orphan drug status by the FDA and The European Commission for the treatment of pulmonary arterial hypertension.

Darusentan is currently in the clinical trail stage of development for hypertension (Nakov R et al, Am J Hypertens 2002, 15: 583).

Despite the beneficial activities of ambrisentan and darusentan, there is a continuing need for new compounds to treat the aforementioned diseases and conditions.

### SUMMARY OF THE INVENTION

This invention relates to novel endothelin receptor antagonists that selectively inhibit the interaction between Endothelin-1 (ET-1) and endothelin type-A receptors, their derivatives, acceptable acid addition salts. This invention also provides compositions comprising a compound of this invention and the use of such compositions in methods of treating diseases and conditions that are beneficially treated by endothelin receptor antagonists, particularly those diseases and conditions that are beneficially treated by selective inhibitors of endothelin type-A receptors.

### DETAILED DESCRIPTION OF THE INVENTION

The terms “ameliorate” and “treat” are used interchangeably and include both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

The term “disease” refers to any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of ambrisentan will inherently contain small amounts of deuterated isotopologues. The concentration of naturally abundant stable hydrogen and carbon isotopes, notwithstanding this variation, is small and immaterial with respect to the degree of stable isotopic substitution of compounds of this invention. See, for instance, Wada E et al., Seikagaku 1994, 66:15; Ganes L Z et al., Comp Biochem Physiol Mol Integr Physiol 1998, 119:725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each deuterium present at a site designated as a potential site of deuteration on the compound of at least 1000 (15% deuterium incorporation), at least 1500 (22.5% deuterium incorporation), at least 2000 (30% deuterium incorporation), at least 2500 (37.5% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 22.5% while the other could be deuterated at 37.5% and still be considered a compound wherein the isotopic enrichment factor is at least 1500 (22.5%).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

The term “isotopologue” refers to a species that differs from a specific compound of this invention only in the isotopic composition thereof or of its ions. The term “compound,” as used herein, is also intended to include solvates or hydrates thereof.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention (e.g., compounds of Formula I), contain an asymmetric carbon atom at the 2 position of the propionic acid backbone, alpha to the carbonyl group. As such, compounds of this invention can exist as either individual enantiomers (e.g., one of 2(S) or 2(R)), or mixtures of the two enantiomers. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates. Other embodiments are those wherein the compound is an isolated compound. The term “at least X % enantiomerically enriched” as used herein means that at least X %, beyond the statistical distribution of a racemic mixture, of the compound is a single enantiomeric form, wherein X is a number between 0 and 100, inclusive.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium.

“Stereoisomer” refers to both enantiomers and diastereomers.

“Tert”, “t”, and “t−” each refer to tertiary.

“US” refers to the United States of America.

“FDA” refers to Food and Drug Administration.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

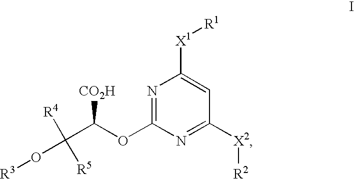

, or a salt thereof; wherein:

each of X¹ and X² is selected independently from a bond and an oxygen atom;

each of R¹, R², and R³ is independently selected from CH₃, CH₂D, CHD₂ and CD₃, provided that at least one of R¹, R², and R³ comprises a deuterium atom; and

each of R⁴ and R⁵ is independently phenyl wherein 1 to 5 hydrogen atoms are optionally replaced with deuterium.

In one embodiment, each of R¹, R² and R³ is independently selected from CH₃ and CD₃.

In another embodiment, R² and R³ are the same.

In another embodiment, each of R⁴ and R⁵ is independently selected from phenyl and C₆D₅.

In another embodiment, R⁴ and R⁵ are the same.

In still another embodiment, X¹ and X² are the same.

In a more specific embodiment, R¹ is CD₃.

In another specific embodiment, each of R¹ and R² is CD₃.

In another specific embodiment, each of R¹, R² and R³ is CD₃.

In another specific embodiment, each of R¹ and R² is CD₃ and each of X¹ and X² is O.

In another specific embodiment, each of R¹, R² and R³ is CD₃ and each of X¹ and X² is O.

In another specific embodiment, each of R⁴ and R⁵ is C₆D₅.

In another specific embodiment, each of R⁴ and R⁵ is C₆D₅ and each of X¹ and X² is O.

In another specific embodiment, R³ is CD₃.

In another specific embodiment, R³ is CD₃ and each of X¹ and X² is O.

In yet another embodiment, the compound is selected from any one of the compounds (Cmpd) set forth in Table 1 (below):

TABLE 1
Exemplary Embodiments of Formula I*

| Cmpd | X¹ | X² | R¹ | R² | R³ | R⁴ | R⁵ |
|---|---|---|---|---|---|---|---|
| 101 | — | — | CD₃ | CD₃ | CH₃ | C₆H₅ | C₆H₅ |
| 102 | — | — | CH₃ | CH₃ | CD₃ | C₆H₅ | C₆H₅ |
| 103 | — | — | CH₃ | CH₃ | CH₃ | C₆D₅ | C₆D₅ |
| 104 | — | — | CD₃ | CD₃ | CH₃ | C₆D₅ | C₆D₅ |
| 106 | — | — | CH₃ | CH₃ | CD₃ | C₆D₅ | C₆D₅ |
| 107 | — | — | CD₃ | CD₃ | CD₃ | C₆H₅ | C₆H₅ |
| 108 | — | — | CD₃ | CD₃ | CD₃ | C₆D₅ | C₆D₅ |
| 109 | O | O | CD₃ | CD₃ | CH₃ | C₆H₅ | C₆H₅ |
| 110 | O | O | CH₃ | CH₃ | CD₃ | C₆H₅ | C₆H₅ |
| 111 | O | O | CH₃ | CH₃ | CH₃ | C₆D₅ | C₆D₅ |
| 112 | O | O | CD₃ | CD₃ | CH₃ | C₆D₅ | C₆D₅ |
| 113 | O | O | CH₃ | CH₃ | CD₃ | C₆D₅ | C₆D₅ |
| 114 | O | O | CD₃ | CD₃ | CD₃ | C₆H₅ | C₆H₅ |
| 115 | O | O | CD₃ | CD₃ | CD₃ | C₆D₅ | C₆D₅ |

*Note: the notation “—” in Table 1 indicates a bond.

In another set of embodiments, any atom not designated as deuterium in any of the embodiments set forth above is present at its natural isotopic abundance.

The synthesis of compounds of Formula I can be readily achieved by synthetic chemists of ordinary skill. Relevant procedures and intermediates are disclosed, for instance in PCT Publication Nos. WO 1996011914 (Ambrisentan) and WO 1996011914 (Darusentan).

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

A convenient method for synthesizing compounds of Formula I is depicted in Scheme 1.

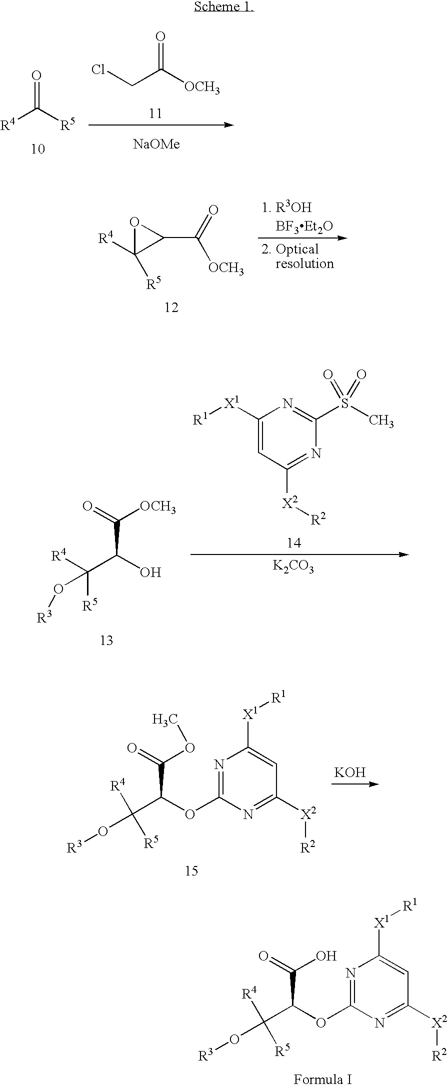

For exemplary purposes within the description of this scheme, R⁴═R⁵=phenyl. Accordingly, as shown in Scheme 1, the condensation of benzophenone 10 with methyl 2-chloroacetate 11 in the presence of NaOMe in THF affords the corresponding 3,3-diphenyloxirane-2-carboxylic acid methyl ester 12, which by treatment with BF₃.Et₂O and methanol, followed by optical resolution, affords the pure enantiomer 2(S)-hydroxy-3-methoxy-3,3-diphenylpropionic acid methyl ester 13. The condensation of 13 with the appropriately deuterated (methylsulfonyl)pyrimidine 14 by means of K₂CO₃ in DMF affords 2-(4,6-dimethylpyrimidin-2-yloxy)-3-methoxy-3,3-diphenylpropionic acid methyl ester 15, which is finally hydrolyzed with KOH in hot dioxane to afford compounds of Formula I. The appropriately deuterated pyrimidine compounds 14 can be synthesized as shown in Schemes 2a and 2b.

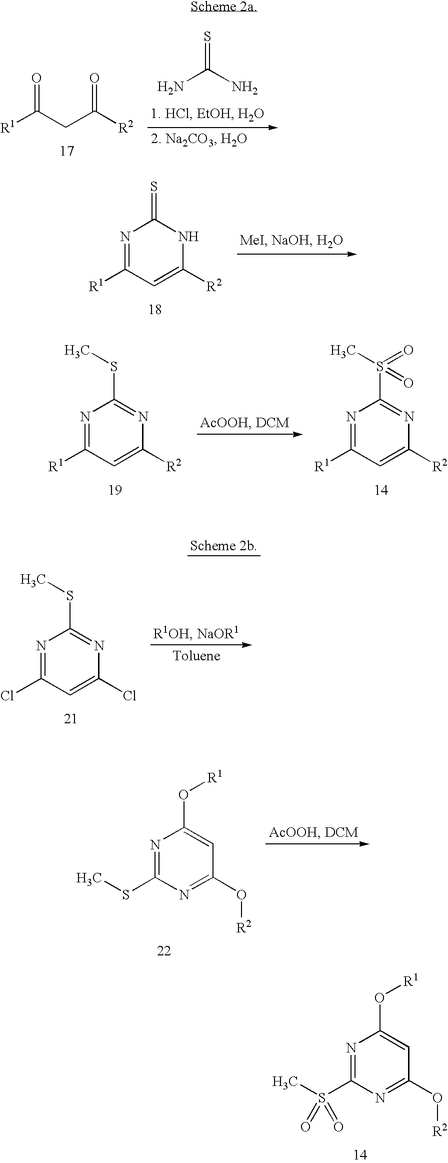

As shown in Scheme 2a, pyrimidine 18 can be prepared from the commercially available pentane-2,4-dione or commercially available d₆-pentane-2,4-dione (17), as described by Parushothaman, E et al, Ind J Het Chem 2001, 11: 43. Pyrimidine 18 can then be converted to S-methylpyrimidine 19 by methylation with methyl iodide, followed by oxidation to the intermediate 14 (wherein X¹ and X² are bonds), as described in WO 2003013545.

As shown in Scheme 2b, commercially available, 4,6-dichloro-2-(methylthio)pyrimidine 21 is reacted with appropriately deuterated methanol to yield the S-methylpyrimidine 22, as described in WO 2002008207, followed by oxidation to the sulfone the intermediate 14 (wherein X¹ and X² are oxygen), as described in WO 2003013545. Variations of the procedures set forth in Scheme 2 and their optimization are within the skill of the ordinary practitioner.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, R⁴, R⁵, X¹, X², etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Methods for optimizing reaction conditions and, if necessary, minimizing competing by-products, are known in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser 's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides compositions comprising an effective amount of a compound of Formula I (e.g., including any of the formulae herein), or a pharmaceutically acceptable salt of said compound; and an acceptable carrier. In one embodiment, the composition is pyrogen-free. In a more specific embodiment, the composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g., Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

Another embodiment of the invention provides an implantable medical device coated with a compound or a composition comprising a compound of this invention, such that said compound is therapeutically active.

Another embodiment of the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with an endothelin antagonist. Such agents are the same as those indicated as being useful in combination with ambrisentan or darusentan, including but not limited to, those described in US published applications US 20040105819; US 20040186083; US 20050101608; US 20050119330; US 20050175667; US 20050124624; US 20060009512; US 20060025463; US 20060030611; US 20060122180; US 20060292213; and US 20070004745.

In one embodiment, the second therapeutic agent is an agent useful in the treatment or prevention of a disease or condition selected from pulmonary arterial hypertension, primary pulmonary hypertension, benign prostatic hyperplasia, lower urinary tract symptoms, erectile dysfunction, chronic obstructive pulmonary disease, chronic pelvic pain syndrome type III, primary dysmenorrhea, pre-eclampsia, thalassemia, skin fibrosis, hypertension, hypoxia-induced pulmonary artery hypertension, interstitial lung disease, scleroderma, idiopathic pulmonary fibrosis, pulmonary hypertension, resistant hypertension, chronic thromboembolic pulmonary hypertension, Eisenmenger's syndrome, high blood pressure, coronary disorders, cardiac insufficiency, renal cerebral ischemia, cardiac infarct, migraine, subarachnoid haemorrhage, Raynaud syndrome, skin cancer, atherosclerosis, sickle cell disease, digital ulcers, abnormal ocular pressure disorders, glaucoma, platelet disorder, hypercoagulation states, thrombocytosis, thrombocythemia, renal disease, renal failure, peripheral vascular disease, stable angina, unstable angina, myocardial infarction, eclampsia, asthma, bronchospastic lung disease, gastrointestinal disorders, obliterative bronchiolitis, lymphangioleiomyomatosis, retinopathy, neuropathy, insulinopathy, edema, endothelial dysfunction, baroreceptor dysfunction, headache, and others, including, but not limited to those disclosed in the following patents and published applications: US 20040105819; US 20040186083; US 20050101608; US 20050119330; US 20050175667; US 20050124624; US 20060009512; US 20060025463; US 20060030611; US 20060122180; US 20060292213; US 20070004745; U.S. Pat. No. 6,635,648; U.S. Pat. No. 5,292,740; U.S. Pat. No. 6,586,391; U.S. Pat. No. 6,869,970; U.S. Pat. No. 5,945,448; U.S. Pat. No. 5,696,116; WO 2005101608; WO 2004082637; WO 2004017993; WO 2002074034; and WO 9638173.

In one embodiment, the second therapeutic agent is selected from a prostacyclin, a prostacyclin derivative, an endothelin antagonist, a dopaminergic agonist, a phosphodiesterase inhibitor, a sympathetic nervous system antagonist, an inhibitor of endothelin converting enzyme, an antihypertensive, an alpha-adrenergic blocker, a HMG-CoA reductase inhibitor, an angiotensin II receptor antagonist, an epidermal growth factor receptor tyrosine kinase inhibitor, an aldosterone receptor antagonist, or a 5-HT_{1B/1D} receptor antagonist.

In another embodiment, the second therapeutic agent is an aldosterone receptor antagonist.

In another embodiment, the second therapeutic agent is the aldosterone receptor antagonist eplerenone.

In another embodiment, the second therapeutic agent is a prostacyclin.

In another embodiment, the second therapeutic agent is a prostacyclin selected from iloprost, treprostinil, and beraprost.

In another embodiment, the second therapeutic agent is a HMG-CoA reductase inhibitor.

In another embodiment, the second therapeutic agent is a HMG-CoA reductase inhibitor selected from atorvastatin, cerivastatin, fluvastatin, lovastatin, mevastatin, pravastatin, pitavastatin, rosuvastatin and simvastatin.

In another embodiment, the second therapeutic agent is a phosphodiesterase inhibitor.

In another embodiment, the second therapeutic agent is a phosphodiesterase inhibitor selected from sildenafil, tadalafil, enoximone, and vardenafil.

In another embodiment, the second therapeutic agent is a 5-HT_{1B/1D} receptor antagonist.

In another embodiment, the second therapeutic agent is a 5-HT_{1B/1D} receptor antagonist, selected from zolmitriptan, sumatriptan, eletriptan, frovatriptan, naratriptan, rizatriptan and almotriptan.

In another embodiment, the invention provides separate dosage forms of a compound of this invention and any of the above-described second therapeutic agent, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardsley, N.Y., 1970, 537.

In certain embodiments, an effective amount of a compound of this invention can range from about 0.0001 mg/kg to about 500 mg/kg, from about 0.001 mg/kg to about 50 mg/kg, from about 0.005 mg/kg to about 30 mg/kg or from about 0.1 mg to about 20 mg.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for ambrisentan.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

Some of the second therapeutic agents described above may act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating the activity of endothelin binding to one or more of its receptors in a cell, comprising contacting a cell with one or more compounds of Formula I herein.

According to another embodiment, the invention provides a method of a disease that is beneficially treated by ambrisentan or darusentan in a subject in need thereof, comprising the step of administering to said subject an effective amount of a compound or a composition of this invention. Such diseases are well known in the art and are disclosed in, but not limited to the following patents and published applications: US 20040105819; US 20040186083; US 20050101608; US 20050119330; US 20050175667; US 20050124624; US 20060009512; US 20060025463; US 20060030611; US 20060122180; US 20060292213; US 20070004745; U.S. Pat. No. 6,635,648; U.S. Pat. No. 5,292,740; U.S. Pat. No. 6,586,391; U.S. Pat. No. 6,869,970; U.S. Pat. No. 5,945,448; U.S. Pat. No. 5,696,116; WO 2005101608; WO 2005077347; WO 2005030187; WO 2004100991; WO 2006014930; WO 2006104870; WO 2004082637; WO 2004032922; WO 2006091862; WO 2006055573; WO 2006031931; WO 2004017993; WO 2002074034; WO 2000021509; WO 1999017756; WO 1999029308; WO 1996011914; and WO 199638173

In another embodiment, the method of this invention is used to treat hypertension in a subject in need thereof. In yet another embodiment, the method of this invention is used to treat a disease or condition in a patient in need thereof selected from resistant hypertension or pulmonary arterial hypertension. Methods delineated herein also include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, the above method of treatment comprises the further step of co-administering to the patient one or more second therapeutic agents. The choice of second therapeutic agent may be made from any second therapeutic agent known to be useful for co-administration with ambrisentan or darusentan. Examples of such agents and the conditions and diseases for which each may be used in conjunction with a compound of this invention are described in the following patents and published applications: US 20040105819; US 20040186083; US 20050101608; US 20050119330; US 20050175667; US 20050124624; US 20060009512; US 20060025463; US 20060030611; US 20060122180; US 20060292213; US 20070004745; U.S. Pat. No. 6,635,648; U.S. Pat. No. 5,292,740; U.S. Pat. No. 6,586,391; U.S. Pat. No. 6,869,970; U.S. Pat. No. 5,945,448; U.S. Pat. No. 5,696,116; WO 2005101608; WO 2005077347; WO 2005030187; WO 2004100991; WO 2006014930; WO 2006104870; WO 2004082637; WO 2004032922; WO 2006091862; WO 2006055573; WO 2006031931; WO 2004017993; WO 2002074034; WO 2000021509; WO 1999017756; WO 1999029308; WO 1996011914; and WO 199638173. More specific examples of second therapeutic agents that may be employed in the methods of this invention are those set forth above for use in combination compositions.

In particular, the combination therapies of this invention include treatment of the following conditions by administering a compound of Formula I together with a second therapeutic agent: pulmonary arterial hypertension, primary pulmonary hypertension, benign prostatic hyperplasia, lower urinary tract symptoms, erectile dysfunction, chronic obstructive pulmonary disease, chronic pelvic pain syndrome type III, primary dysmenorrhea, pre-eclampsia, thalassemia, skin fibrosis, hypertension, hypoxia-induced pulmonary artery hypertension, interstitial lung disease, scleroderma, idiopathic pulmonary fibrosis, pulmonary hypertension, resistant hypertension, chronic thromboembolic pulmonary hypertension, Eisenmenger's syndrome, high blood pressure, coronary disorders, cardiac insufficiency, renal cerebral ischemia, cardiac infarct, migraine, subarachnoid haemorrhage, Raynaud syndrome, skin cancer, atherosclerosis, sickle cell disease, digital ulcers, abnormal ocular pressure disorders, glaucoma, platelet disorder, hypercoagulation states, thrombocytosis, thrombocythemia, renal disease, renal failure, peripheral vascular disease, stable angina, unstable angina, myocardial infarction, eclampsia, asthma, bronchospastic lung disease, gastrointestinal disorders, obliterative bronchiolitis, lymphangioleiomyomatosis, retinopathy, neuropathy, insulinopathy, edema, endothelial dysfunction, baroreceptor dysfunction, headache, and others, including, but not limited to those disclosed in the following patents and published applications: 20040105819; 20040186083; 20050101608; 20050119330; 20050175667; 20050124624; 20060009512; 20060025463; 20060030611; 20060122180; 20060292213; 20070004745; U.S. Pat. No. 6,635,648; U.S. Pat. No. 5,292,740; U.S. Pat. No. 6,586,391; U.S. Pat. No. 6,869,970; U.S. Pat. No. 5,945,448; U.S. Pat. No. 5,696,116; WO 2005101608; WO 2004082637; WO 2004017993; WO 2002074034; and WO 9638173A1.

In yet another embodiment, the combination therapies of this invention include treatment of hypertension by administering a compound of Formula I together with a second therapeutic agent. In another embodiment, the combination therapies of this invention include treatment of resistant hypertension and pulmonary hypertension by administering a compound of Formula I together with a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of Formula I for use in the treatment or prevention in a subject of a disease, disorder or symptom thereof delineated herein.

### Diagnostic Methods and Kits

The compounds and compositions of this invention are also useful as reagents in methods for determining the concentration of ambrisentan or darusentan in solution or biological sample such as plasma, examining the metabolism of ambrisentan or darusentan and other analytical studies.

According to one embodiment, the invention provides a method of determining the concentration, in a solution or a biological sample, of ambrisentan or darusentan, comprising the steps of:

a) adding a known concentration of a compound of Formula I to the solution of biological sample;

b) subjecting the solution or biological sample to a measuring device that distinguishes ambrisentan or darusentan from a compound of Formula I;

c) calibrating the measuring device to correlate the detected quantity of the compound of Formula I with the known concentration of the compound of Formula I added to the biological sample or solution; and

d) measuring the quantity of ambrisentan or darusentan in the biological sample with said calibrated measuring device; and

e) determining the concentration of ambrisentan or darusentan in the solution of sample using the correlation between detected quantity and concentration obtained for a compound of Formula I.

Measuring devices that can distinguish ambrisentan or darusentan from the corresponding compound of Formula I include any measuring device that can distinguish between two compounds that differ from one another only in isotopic abundance. Exemplary measuring devices include a mass spectrometer, NMR spectrometer, or IR spectrometer.

In another embodiment, the invention provides a method of evaluating the metabolic stability of a compound of Formula I comprising the steps of contacting the compound of Formula I with a metabolizing enzyme source for a period of time and comparing the amount of the compound of Formula I with the metabolic products of the compound of Formula I after the period of time.

In a related embodiment, the invention provides a method of evaluating the metabolic stability of a compound of Formula I in a patient following administration of the compound of Formula I. This method comprises the steps of obtaining a serum, urine or feces sample from the patient at a period of time following the administration of the compound of Formula I to the subject; and comparing the amount of the compound of Formula I with the metabolic products of the compound of Formula I in the serum, urine or feces sample.

The present invention also provides kits for use to treat hypertension (e.g., resistant hypertension or pulmonary hypertension). These kits comprise (a) a pharmaceutical composition comprising a compound of Formula I or a salt thereof, wherein said pharmaceutical composition is in a container; and (b) instructions describing a method of using the pharmaceutical composition to treat hypertension (e.g., resistant hypertension or pulmonary arterial hypertension).

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In on embodiment, the container is a blister pack.

The kit may additionally comprise a memory aid of the type containing information and/or instructions for the physician, pharmacist or subject. Such memory aids include numbers printed on each chamber or division containing a dosage that corresponds with the days of the regimen which the tablets or capsules so specified should be ingested, or days of the week printed on each chamber or division, or a card which contains the same type of information. For single dose dispensers, memory aids further include a mechanical counter which indicates the number of daily doses that have been dispensed and a battery-powered micro-chip memory coupled with a liquid crystal readout and/or audible reminder signal which, for example, reads out the date that the last daily dose has been taken and/or reminds one when the next dose is to be taken. Other memory aids useful in such kits are a calendar printed on a card, as well as other variations that will be readily apparent.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

### EXAMPLES

### Example 1

### Synthesis of (S)-1-(4-Chlorophenyl)ethanaminium (S)-2-hydroxyl 3-(methoxy-d₃)-3,3-diphenylpropanoate 13b

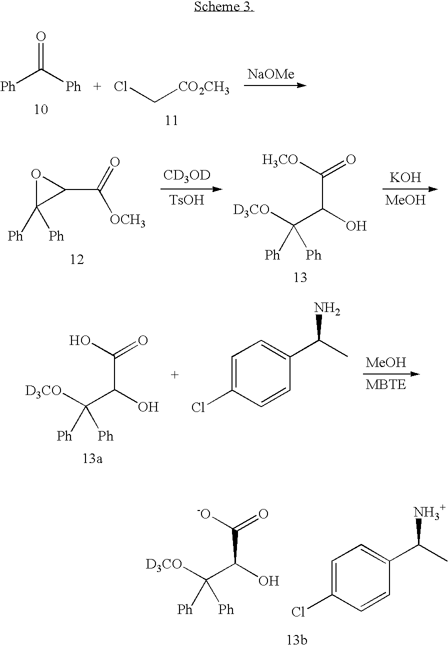

Intermediate 13b was prepared according to Scheme 3, as described below.

Methyl 3,3-diphenyloxirane-2-carboxylate (12). To suspension of sodium methoxide (104.0 g, 1.92 mol) in tetrahydrofuran (340 mL) at 0° C. under sonication was added a solution of benzophenone 10 (200.0 g, 1.10 mol) and methyl chloroacetate 11 (208.0 g, 1.92 mol) in tetrahydrofuran (40 mL) over 4 hours (hr). The reaction vessel temperature was maintained at 0° C. for the 4 hr period and after addition was complete the mixture was stirred/sonicated at 0° C. for an additional 1 hr. The resulting reaction mixture was poured into water (700 mL) and the aqueous mixture extracted with methyl tert-butyl ether (MTBE; 2×700 mL). The organic solution was dried over sodium sulfate (Na₂SO₄), filtered and the solvent removed under reduced pressure to give 230 g of crude product as a dark solid. A portion (23 g) of the crude product was purified by Medium Pressure Liquid Chromatography (MPLC), eluting with a gradient of 0-100% ethyl acetate/hexanes to give 21 g of the product 12 as a colorless oil that solidified on standing. ¹H-NMR (300 MHz, CDCl₃): δ 3.45 (s, 3H), 3.95 (s, 1H), 7.30-7.51 (m, 10H). LCMS (method: Luna 3μ C₁₈ column, 20×4 mm; 2-95% acetonitrile/water with 0.1% formic acid in 3.3 min with 1.7 min hold; Wavelength: 254 nm; API-ES, positive): retention time: 3.60 min; (M+Na⁺): 277.1.

Methyl 2-hydroxyl 3-(methoxy-d₃)-3,3-diphenylpropanoate (13). To a solution of compound 12 (20 g, 78 mmol) in methanol-d₄ (65 mL) was added p-toluenesulfonic acid, monohydrate (0.144 g, 1 mol %) and the solution stirred at room temperature for 2 days. The reaction mixture was concentrated under reduced pressure to give 22 g (97%) of the product 13 as a white solid. ¹H-NMR (300 MHz, CDCl₃): δ 3.58 (s, 3H), 5.19 (s, 1H), 7.30-7.42 (m, 10H). LCMS (method: Luna 3μ C₁₈ column, 20×4 mm; 2-95% acetonitrile/water with 0.1% formic acid in 3.3 min with 1.7 min hold; Wavelength: 254 nm; API-ES, positive): retention time: 3.39 min; (M+Na⁺): 312.2.

2-Hydroxyl 3-(methoxy-d₃)-3,3-diphenylpropanoic acid (13a). To a solution of compound 13 (22 g, 78 mmol) in methanol (200 mL) was added potassium hydroxide (90 g, 1.6 mol) with stirring. The reaction mixture was stirred for 2 hr at room temperature then concentrated under reduced pressure to a residue which was diluted with water (1 L) and MTBE (1.5 L). The mixture was acidified by addition of 10% sulfuric acid (2.2 L). The organic phase was separated and the aqueous phase extracted with MTBE (3×500 mL). The combined organic extracts were dried (Na₂SO₄), filtered and the solvent removed under reduced pressure to give 16.3 g (78%) of product 13a as a white solid. ¹H-NMR (300 MHz, CDCl₃): δ 3.40 (bs, 1H), 5.08 (s, 1H), 7.35-7.48 (m, 10H). Chiral HPLC (method: ChiraPak AD column, 250×4.6 mm, isocratic method 90% hexanes: 10% ethanol, 0.6 mL/min over 20 min; Wavelength: 220 nm): retention time: 4.95 and 5.92 min. LCMS (method: Luna 3μ C₁₈ column, 20×4 mm; 2-95% acetonitrile/water with 0.1% formic acid in 3.3 min with 1.7 min hold; Wavelength: 254 nm; API-ES, positive): retention time: 3.12 min; (M+Na⁺): 298.1.

(S)-1-(4-Chlorophenyl)ethanaminium (S)-2-hydroxyl 3-(methoxy-d₃)-3,3-diphenylpropanoate (13b). To a solution of 13a (2 g, 7.2 mmol) in methanol (4.8 mL) and MTBE (7 mL) under reflux conditions was added (S)-1-(4-chlorophenyl)ethanamine (0.5 mL, 14.4 mmol) with stirring. After 5 min of stirring under reflux conditions, the mixture was cooled to 0° C. The precipitated salt was filtered, washed with MTBE (20 mL) and dried to give 1.5 g (48%) of the salt 13b as a white solid. ¹H-NMR (300 MHz, CD₃OD): δ 1.57 (d, 3H), 4.40 (q, 1H), 4.94 (s, 1H), 7.19-7.32 (m, 6H), 7.40-7.45 (m, 8H). Chiral HPLC (method: ChiraPak AD column, 250×4.6 mm, isocratic method 90% hexanes: 10% ethanol, 0.6 mL/min over 20 min; Wavelength: 230 nm): retention time: 4.91 min.

### Example 2

### 4,6-(Dimethoxy-d₃)-2-(methylsulfonyl)pyrimidine 14

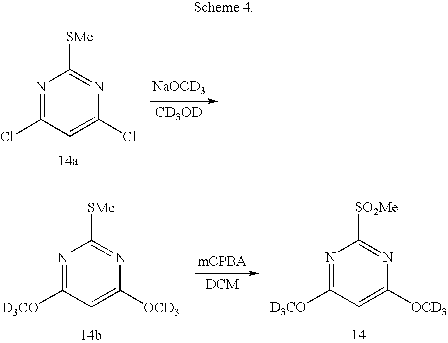

Intermediate 14 was prepared according to Scheme 4, as described below.

4,6-(Dimethoxy-d₃)-2-(methylthio)pyrimidine (14b). Sodium (2.36 g, 0.102 mol) was added portionwise at 0° C. to methanol-d₄ (50 mL) and the mixture stirred until all sodium had dissolved. 4,6-Dichloro-2-methylthiopyrimidine 14a (5.00 g, 0.256 mol) was added at 0° C., the mixture was allowed to warm to room temperature, then was stirred overnight (11 hr). The resulting mixture was poured into water (200 mL) and extracted with ethyl acetate (300 mL). The organic phase was washed with brine (100 mL), dried (Na₂SO₄), filtered and the solvent removed under reduced pressure to give 5.0 g of light orange solid. The crude product was purified by MPLC, eluting with dichloromethane to give 1.97 g of the product 14b as a white solid. ¹H-NMR (300 MHz, DMSO-d₆): δ 2.50 (peak obscured by DMSO), 5.93 (s, 1H). LCMS (method: Luna 3μ C₁₈ column, 20×4 mm; 2-95% acetonitrile/water with 0.1% formic acid in 3.3 min with 1.7 min hold; Wavelength: 254 nm; API-ES, positive): retention time: 3.32 min; (M+H⁺): 193.2.

4,6-(Dimethoxy-d₃)-2-(methylsulfonyl)pyrimidine (14). To a solution of 14b (1.92 g, 10 mmol) in dichloromethane (150 mL) at ˜0° C. was added gradually a solution of ˜77% m-chloroperoxybenzoic acid (4.72 g, 21 mmol) in dichloromethane (50 mL). The reaction mixture was allowed to warm to room temperature then was stirred 4.25 hr. The resulting mixture was washed with saturated sodium bicarbonate solution (200 m)—effervescence was observed, was dried (Na₂SO₄), filtered and the solvent removed under reduced pressure to give 2.41 g of white solid. The crude product was purified by MPLC, eluting with a gradient of 0-100% ethyl acetate/hexanes to give 1.97 g of the product 14 as a white solid. ¹H-NMR (300 MHz, CDCl₃): δ 3.36 (s, 3H), 6.18 (s, 1H). LCMS (method: Luna 3 C₁₈ column, 20×4 mm; 2-95% acetonitrile/water with 0.1% formic acid in 3.3 min with 1.7 min hold; Wavelength: 254 nm; API-ES, positive): retention time: 2.38 min; (M+H⁺): 225.0.

### Example 3

### (S)-2-[(4,6-dimethoxy-d₆)pyrimidinyl-2-yloxy]-3-(methoxy-d₃)-3,3-diphenylpropanoic acid (Compound 114)

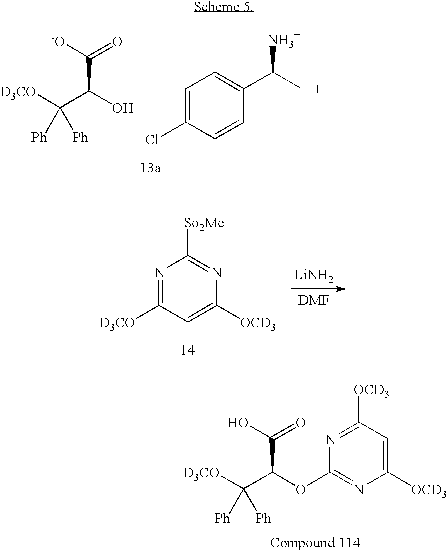

Compound 114 was prepared according to Scheme 5, as described below.

(S)-2-[(4,6-dimethoxy-d₆)pyrimidinyl-2-yloxy]-3-(methoxy-d₃)-3,3-diphenylpropanoic acid (Compound 114). To a suspension of 13a (2.05 g, 4.77 mmol) in N,N-dimethylformamide (7 mL) at room temperature was added lithium amide (0.33 g, 14.32 mmol) and the mixture was stirred 10 min. A solution of 14 (1.39 g, 6.20 mmol) in N,N-dimethylformamide (5 mL) was added dropwise over 2 minutes and the reaction mixture was stirred at room temperature overnight. The resulting mixture was diluted with water (25 mL) and ethyl acetate (50 mL), then was acidified with 10% sulfuric acid (11 mL). The organic phase was separated, washed with water (20 mL), dried (Na₂SO₄), filtered and the solvent removed under reduced pressure to give crude product as a colorless oil. The crude oil was crystallized from ethyl acetate/hexanes to give 0.828 g of Compound 114 as a white solid after drying in a vacuum oven for 1 hr at 50° C. The material contained residual DMF by ¹H NMR. ¹H-NMR (300 MHz, CDCl₃): δ 5.73 (s, 1H), 6.20 (s, 1H), 7.27-7.41 (m, 8H), 7.50-7.53 (m, 2H). LCMS (method: Luna 3μ C₁₈ column, 20×4 mm; 2-95% acetonitrile/water with 0.1% formic acid in 3.3 min with 1.7 min hold; Wavelength: 254 nm; API-ES, negative): retention time: 3.68 min; (M−1): 418.2.

### Example 3

### Evaluation of Metabolic Stability

Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

Experimental Procedures: Human liver microsomes are obtained from a commercial source (e.g., XenoTech, LLC (Lenexa, Kans.)). The incubation mixtures are prepared as follows:

Reaction Mixture Composition

| | | |
|---|---|---|
| Liver Microsomes | 0.5-2.0 | mg/mL |
| NADPH | 1 | mM |
| Potassium Phosphate, pH 7.4 | 100 | mM |
| Magnesium Chloride | 10 | mM |
| Test Compound | 0.1-1 | μM. |

Incubation of Test Compounds with Liver Microsomes: The reaction mixture, minus cofactors, is prepared. An aliquot of the reaction mixture (without cofactors) is incubated in a shaking water bath at 37° C. for 3 minutes. Another aliquot of the reaction mixture is prepared as the negative control. The test compound is added into both the reaction mixture and the negative control at a final concentration of 1 μM. An aliquot of the reaction mixture is prepared as a blank control, by the addition of plain organic solvent (not the test compound). The reaction is initiated by the addition of cofactors (not into the negative controls), and then incubated in a shaking water bath at 37° C. Aliquots (200 μL) are withdrawn in triplicate at multiple time points (e.g., 0, 15, 30, 60, and 120 minutes) and combined with 800 μL of ice-cold 50/50 acetonitrile/dH₂O to terminate the reaction. The positive controls, testosterone and propranolol, as well as ambrisentan or darusentan, are each run simultaneously with the test compounds in separate reactions.

All samples are analyzed using LC-MS (or MS/MS). An LC-MRM-MS/MS method is used for metabolic stability. Also, Q1 full scan LC-MS methods are performed on the blank matrix and the test compound incubation samples. The Q1 scans serve as survey scans to identify any sample unique peaks that might represent the possible metabolites. The masses of these potential metabolites can be determined from the Q1 scans.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a test compound in 100 mM potassium phosphate buffer (pH 7.4) was incubated at 37° C. in triplicate. Positive controls contain 1 μM of ambrisentan or darusentan instead of a test compound of. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

What is claimed is:

1. A compound of Formula I: 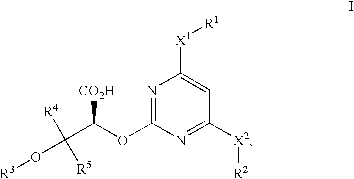
, or a pharmaceutically acceptable salt thereof; wherein:
each of X¹ and X² is independently selected from a bond and an oxygen atom;
each of R¹, R², and R³ is independently selected from CH₃, CH₂D, CHD₂ and CD₃, provided that at least one of R¹, R², and R³ comprises a deuterium atom; and
each of R⁴ and R⁵ is independently phenyl wherein 1 to 5 hydrogen atoms are optionally replaced with deuterium.

2. The compound of claim 1, wherein each of R¹, R² and R³ is independently selected from CH₃ and CD₃.

3-5. (canceled)

6. The compound of any one of claims 1, 2 and 16-29, wherein X¹ and X² are the same.

7. The compound of claim 1, selected from any one of the compounds set forth in the table below:

| Cmpd | X¹ | X² | R¹ | R² | R³ | R⁴ | R⁵ |
|---|---|---|---|---|---|---|---|
| 101 | — | — | CD₃ | CD₃ | CH₃ | C₆H₅ | C₆H₅ |
| 102 | — | — | CH₃ | CH₃ | CD₃ | C₆H₅ | C₆H₅ |
| 103 | — | — | CH₃ | CH₃ | CH₃ | C₆D₅ | C₆D₅ |
| 104 | — | — | CD₃ | CD₃ | CH₃ | C₆D₅ | C₆D₅ |
| 106 | — | — | CH₃ | CH₃ | CD₃ | C₆D₅ | C₆D₅ |
| 107 | — | — | CD₃ | CD₃ | CD₃ | C₆H₅ | C₆H₅ |
| 108 | — | — | CD₃ | CD₃ | CD₃ | C₆D₅ | C₆D₅ |
| 109 | O | O | CD₃ | CD₃ | CH₃ | C₆H₅ | C₆H₅ |
| 110 | O | O | CH₃ | CH₃ | CD₃ | C₆H₅ | C₆H₅ |
| 111 | O | O | CH₃ | CH₃ | CH₃ | C₆D₅ | C₆D₅ |
| 112 | O | O | CD₃ | CD₃ | CH₃ | C₆D₅ | C₆D₅ |
| 113 | O | O | CH₃ | CH₃ | CD₃ | C₆D₅ | C₆D₅ |
| 114 | O | O | CD₃ | CD₃ | CD₃ | C₆H₅ | C₆H₅ |
| 115 | O | O | CD₃ | CD₃ | CD₃ | C₆D₅ | C₆D₅ |

, wherein “—” represents a bond.
, wherein “—” represents a bond.

8. A pyrogen-free composition comprising a compound of Formula I: 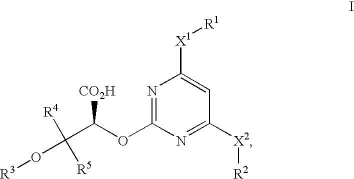
, or a pharmaceutically acceptable salt thereof: wherein:
each of X¹ and X² is independently selected from a bond and an oxygen atom;
each of R¹, R², and R³ is independently selected from CH₃, CH₂D, CHD₂ and CD₃, provided that at least one of R¹, R², and R³ comprises a deuterium atom; and
each of R⁴ and R⁵ is independently phenyl wherein 1 to 5 hydrogen atoms are optionally replaced with deuterium, and an acceptable carrier.

9. The composition of claim 8, formulated for pharmaceutical administration, wherein the carrier is a pharmaceutically acceptable carrier.

10. The composition of claim 9 in an oral formulation.

11. The composition of claim 9 or 10, additionally comprising a second therapeutic agent selected from a prostacyclin, a prostacyclin derivative, an endothelin antagonist, a dopaminergic agonist, a phosphodiesterase inhibitor, a sympathetic nervous system antagonist, an inhibitor of endothelin converting enzyme, an antihypertensive, an alpha-adrenergic blocker, a HMG-CoA reductase inhibitor, an angiotensin II receptor antagonist, an epidermal growth factor receptor tyrosine kinase inhibitor, an aldosterone receptor antagonist, and a 5-HT_{1B/1D} receptor antagonist.

12. A method of treating hypertension in a subject in need thereof comprising the step of administering to the subject a therapeutically effective amount of a composition comprising a compound of Formula I: 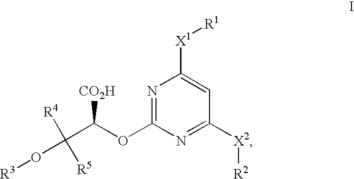
, or a pharmaceutically acceptable salt thereof; wherein:
each of X¹ and X² is independently selected from a bond and an oxygen atom;
each of R¹, R², and R³ is independently selected from CH₃, CH₂D, CHD₂ and CD₃, provided that at least one of R¹, R², and R³ comprises a deuterium atom; and
each of R⁴ and R⁵ is independently phenyl wherein 1 to 5 hydrogen atoms are optionally replaced with deuterium, and a pharmaceutically acceptable carrier.

13. The method of claim 12, wherein the hypertension is pulmonary arterial hypertension.

14. The method of claim 12 or 13, additionally comprising the step of co-administering to said patient a second therapeutic agent selected from a prostacyclin, a prostacyclin derivative, an endothelin antagonist, a dopaminergic agonist, a phosphodiesterase inhibitor, a sympathetic nervous system antagonist, an inhibitor of endothelin converting enzyme, an antihypertensive, an alpha-adrenergic blocker, a HMG-CoA reductase inhibitor, an angiotensin II receptor antagonist, an epidermal growth factor receptor tyrosine kinase inhibitor, an aldosterone receptor antagonist, and a 5-HT_{1B/1D} receptor antagonist.

15. The method of claim 14, wherein the second therapeutic agent is useful to treat hypertension.

16. The compound of claim 1, wherein R² and R³ are the same.

17. The compound of claim 2, wherein R² and R³ are the same.

18. The compound of claim 1, wherein each of R⁴ and R⁵ is independently selected from phenyl and C₆D₅.

19. The compound of claim 2, wherein each of R⁴ and R⁵ is independently selected from phenyl and C₆D₅.

20. The compound of claim 16, wherein each of R⁴ and R⁵ is independently selected from phenyl and C₆D₅.

21. The compound of claim 17, wherein each of R⁴ and R⁵ is independently selected from phenyl and C₆D₅.

22. The compound of claim 1, wherein R⁴ and R⁵ are the same.

23. The compound of claim 2, wherein R⁴ and R⁵ are the same.

24. The compound of claim 16, wherein R⁴ and R⁵ are the same.

25. The compound of claim 17, wherein R⁴ and R⁵ are the same.

26. The compound of claim 18, wherein R⁴ and R⁵ are the same.

27. The compound of claim 19, wherein R⁴ and R⁵ are the same.

28. The compound of claim 20, wherein R⁴ and R⁵ are the same.

29. The compound of claim 21, wherein R⁴ and R⁵ are the same.
